(19) United States
(12) Patent Application Publication
(10) Pub. No.: US 2006/0030027 A1
(43) Pub. Date: Feb. 9, 2006

(54) Capture and removal of biomolecules from body fluids using partial molecular imprints

(75) Inventors: Richard N. Ellson, Palo Alto, CA (US); Mitchell W. Mutz, Palo Alto, CA (US)

Correspondence Address: REED INTELLECTUAL PROPERTY LAW GROUP, 1400 PAGE MILL ROAD, PALO ALTO, CA, 94304-1124, US

(73) Assignee: Aspira Biosystems, Inc., Burlingame, CA (US)

(21) Appl. No.: 11/198,045

(22) Filed: Aug. 4, 2005

Related U.S. Application Data
(60) Provisional application No. 60/599,284, filed on Aug. 4, 2004.

(51) Int. Cl. C12M 1/34; A61K 39/395

(52) U.S. Cl. 435/287.1; 424/140.1

(57) ABSTRACT

The invention provides methods and devices for the capture and removal of target biomolecules from a patient's body fluid, particularly blood or a blood component, using a partial imprint material. The partial imprint material is composed of a matrix composition having partial imprint cavities that correspond to a segment of a target biomolecule but which are capable of removing the entire target biomolecule from the body fluid. The method can be implemented by removing a volume of a patient's body fluid, e.g., blood, bringing the body fluid or a component thereof into contact with the partial imprint material under conditions effective to capture the target biomolecule, and returning the body fluid to the patient. A modified dialysis or apheresis device can be used in which the body fluid is removed, continuously passed through a circuit containing the partial imprint material, and re-introduced into the patient's body following treatment.

### CROSS-REFERENCE TO RELATED APPLICATIONS

This application claims priority under 35 U.S.C. §119(e)(1) to Provisional U.S. Patent Application Ser. No. 60/599,284, filed Aug. 4, 2004. The disclosure of the aforementioned provisional patent application is incorporated by reference herein.

### TECHNICAL FIELD

The invention relates generally to the treatment of diseases, disorders, and other adverse medical conditions using a “partial imprint” material. More specifically, the invention pertains to the use of partial imprints of biomolecules associated with a particular disease, disorder, or other adverse medical condition to capture the entire biomolecule and allow removal thereof from a patient's body fluid. The invention is particularly suited for use with an apparatus in which treatment of the body fluid is extracorporeal.

### BACKGROUND

Many diseases result from the presence or overabundance of a particular biomolecule in the body. The biomolecule essentially serves as a pathological effector by triggering a physiological response that directly or indirectly manifests as a disease state. Autoimmune diseases, resulting from a dysfunction of the immune system in which the body attacks its own organs, tissues, and cells, are representative of such diseases. That is, the basic function of the immune system is to recognize and eliminate foreign substances from the body by reaction between a foreign substance and antibodies that are formed in response to the presence of the substance. In certain instances, disturbances can occur which can lead to pathogenic disorders such as, for example, an uncontrolled immune response (i.e., an allergic response) or an abnormal response in which an endogenous substrate is incorrectly recognized as a foreign substance, triggering the production of antibodies thereto, termed “autoantibodies” (i.e., autoimmune disease). It is these autoantibodies that attack endogenous substrates within the body.

Autoimmunity can affect almost every part of the body. Representative autoimmune diseases can involve the adrenal glands, gastric mucosa, pancreas, thyroid, exocrine glands, and red blood cells: Addison's disease is associated with adrenal autoimmunity; Crohn's disease is an inflammatory bowel disease triggered by an autoimmune response; insulin-dependent diabetes mellitus (IDDM) involves the autoimmune destruction of insulin-producing cells in the pancreas via an autoimmune response; Grave's disease and Hashimoto's thyroiditis are autoimmune disease of the thyroid (in Grave's disease, thyroglobulin and thyroid peroxidase autoantibodies are generated); myasthenia gravis involves an antibody-mediated immune attack directed against acetylcholine receptors (AChRs) at neuromuscular junctions; autoimmune gastritis and pernicious anemia are associated with autoantibodies to gastric parietal cells; multiple sclerosis involves the production of autoantibodies that invade the central nervous system; Sjögren's syndrome involves the autoimmune destruction of the exocrine glands; and autoimmune hemolytic anemia and thrombocytopenia purpura involve the generation of autoantibodies to “antigens” on the red cell membrane and platelet membrane, respectively. There are also a number of cutaneous autoimmune diseases, including psoriasis, pemphigus vulgarus, bullous pemphigoid, epidermolysis bullosa acquisita, and cutaneous lupus erythematosis. Autoimmune diseases can also be systemic in nature; such diseases include systemic lupus erythematosis (SLE), scleroderma (systemic sclerosis), rheumatoid arthritis (RA), and antiphospholipid/cofactor syndromes. Autoimmune diseases are also associated with many behavioral disorders and involve complex mechanisms that are believed to affect various regions of the brain, including Sydenham's Chorea, Tourette's Syndrome, Obsessive-Compulsive Disorder (OCD), pediatric autoimmune disorder associated with streptococchal antibody (PANDAS), and attention-deficit hyperactive disorder (ADHD).

A variety of disorders and diseases that do not involve an autoimmune response are also caused by the presence or overabundance of certain biomolecules, many of which are proteins and peptides. For instance, most forms of Parkinson's disease are caused by an excess of the protein α-synuclein, Alzheimer's disease is associated with the formulation of extracellular and cerebrovascular senile plaques formed from amyloid peptides, particularly β-amyloid, and transmissible spongiform encephalopathies (“prion diseases”) are characterized by abnormal protein deposits in brain tissue. Still other disorders and diseases are associated with an excess of certain types of lipids, e.g., sterols, sterol esters, and triglycerides, and the corresponding lipoproteins that serve to transport such molecules (e.g., high-density lipoproteins, or “HDL”).

Removal of disease-inducing biomolecules from the body has proven to be an effective method of treating many diseases, including renal diseases such as glomerulonephritis, glomerulosclerosis, and nephrotic syndrome. See Harada et al. (1998) Therapeutic Apheresis 2:193-198. It has also been suggested that neoplastic diseases can be treated by removal of a specific cell line, an immunosuppressant blocking factor, or other pathological effectors. See, e.g., U.S. Pat. No. 4,687,808.

Ideally, any process used to remove disease-inducing biomolecules from the body should not only be therapeutically effective but also be free of any significant side effects. Additionally, it would of course be desirable if the method were capable of specifically targeted the disease-inducing biomolecule, so as to prevent removal of endogenous, non-pathogenic species. It would furthermore be desirable to provide a method whose efficacy could easily be monitored during the removal process, and that could be carried out using a simple apparatus that would be cost-effective to produce and straightforward to use in the therapeutic context.

Removal of disease-inducing biomolecules from the body has been carried out using both physical and chemical methods. Physical apheresis techniques do not involve chemical interaction between a disease-inducing biomolecule and a capturing moiety, but rather remove biomolecules according to size and/or molecular weight using a filter. The filter enables a relatively high molecular weight biomolecule to be filtered out of a patient's blood, with the blood then reinfused into the patient. The binding species is fixed in the matrix of a column through which the blood is circulated. For example, U.S. Pat. No. 6,551,266 to Davis Jr. describes the RheoFilter® device, a physical apheresis system that preferentially captures biomolecules larger than 250 Angstroms or having a molecular weight greater than about 500,000 Daltons. Disease-inducing biomolecules in this size and mass range, many of which are associated with vascular disease or risk factors, are stated to include most isoforms of LDL-C, LDL-ox, fibrinogen, IgM, alpha-2 macroglobulin, lipoprotein A, apolipoprotein B, von Willebrand factor, and vitronectin.

In chemical apheresis, techniques are used to bind a disease-inducing biomolecule via a chemical reaction such as that between an antibody and an antigen. Chemical apheresis involves contacting a body fluid containing a particular disease-inducing biomolecule with a capture material that chemically binds the biomolecule and therefore enables removal from the body fluid. For example, U.S. Pat. No. 4,375,414 to Strahilevitz describes an immunoadsorption treatment system for removing biomolecules such as haptens, antigens, and antibodies from the blood using a binding species that chemically binds the biomolecule. U.S. Pat. No. 4,215,688 to Terman et al. describes a method and device for the extracorporeal treatment of disease which involves withdrawing whole blood from a patient, separating the plasma from the whole blood, passing the plasma through a cylinder containing microspheres or beads to which immuno-adsorbent agents are attached, recombining the treated plasma with the remainder of the whole blood, and reinfusing the whole blood into the patient. U.S. Pat. No. 4,381,004 to Babb describes a treatment for infectious or parasitic disease based on extracorporeal processing of whole blood or plasma in order to inactivate the disease-causing microorganisms therein.

Another method of immunadsorption involves the use of microbial ligands, as described in U.S. Pat. No. 4,614,513 to Bensinger. In one embodiment, Bensinger describes removing whole blood from a patient, separating the blood into plasma and cellular components, treating the plasma by passing it through an immunoadsorbent material composed of an inert support having a microbial ligand such as Staphylococcus Protein A covalently bound thereto. The treated plasma is recombined with the cellular components and then returned to the patient.

In U.S. Pat. No. 4,685,900 to Honard et al., a method is described for the treatment of a patient by passing body fluid withdrawn from the patient through a chamber containing a non-radiation biospecific polymer grafted onto and extending away from a support via a spacer moiety. The biospecific polymer, e.g., a hydrogel, is intended to chemically bind specific pathological effectors and thereby remove the pathological effectors from the body fluid. Following treatment of the body fluid in the chamber, the treated body fluid is returned to the patient. The stated purpose of the system was to improve specificity relative to previously described apheresis methods and devices, which the patent characterizes as “semispecific” in that vital low molecular weight components of the body fluid were removed along with the pathological effector.

An example of a commercially available system for the extracorporeal treatment of blood via immunoabsorption is a device containing an Ig-Therasorb® column (Plasma Select), which is composed of a matrix having antibodies covalently bound thereto which are capable of binding human immunoglobulins. U.S. Pat. No. 6,030,614 to Muller-Derlich et al. describes the use of the system in preventing or ameliorating hyperacute or acute rejection of a human donor organ transplanted to a human recipient. That is, the patent describes the use of the system to capture and remove cytotoxic anti-Human Leukocyte Antigens antibodies (anti-HLA antibodies) that a transplant recipient produces against the tissue of the donor organ.

The above methods, both physical and chemical, have disadvantages. In general, physical methods lack specificity since capture is based on a molecular weight threshold. This limits applicability to disease-inducing macromolecules of higher molecular weight. Physical methods are also problematic in that higher molecular weight beneficial molecules are also removed. For chemical apheresis, the development of a the capture substrate chemistry is difficult and may also suffer from a lack of specificity. The manufacture of the capture substrate is also a complex and relatively expensive process due to the nature of producing the modified sheep antibodies.

What is needed is a safe, effective, and convenient method for high-specificity capture and removal of biomolecules, and in particular, antibodies that cause autoimmune responses in individuals who exhibit symptoms of autoimmune disease. It would also be desirable to extend this high-specificity capture to other disease-inducers such as blood-borne pathogens. For example, bacteremia or sepsis is an overwhelming bacterial infection which claims an estimated 250,000 lives per year in the United States. Prior treatments have proven ineffective or prone to deleterious side effects, such as methods based on activated Protein C.

An attractive alternative to traditional therapeutics such as antibiotics or immunotherapy is provided by molecular imprinting (see Zimmerman et al. (2002), “Synthetic Hosts by Monomolecular Imprinting Inside Dendrimers,” Nature 418:399-403 and Zimmerman et al. (2003), “Molecular Imprinting Inside Dendrimers,” J. Am. Chem. Soc. 125(44):13504-18. A molecular imprint material contains cavities that correspond to the three-dimensional structure of a biomolecule to be captured. To prepare a molecular imprint material, a matrix is formed around an entire template molecule identical in structure to the biomolecule to be captured. After the matrix has formed and the template molecule has been removed, the resulting molecular imprint material can be used to selectively capture the template molecule. As early as 1949, a silica gel was created that selectively bound a dye (Dickey (1949) Proc. Natl. Acad. Sci. USA 35:227-229). More recently, an imprint prepared with phenyl-α-D-mannopyranoside was used to resolve a racemic mixture of the saccharide (Wulff (1998) Chemtech 28:19-26). Current molecular imprinting methods use an imprint material composed of an organic polymer (Wulff, supra). The material is formed by covalently or noncovalently binding polymerizable molecules to a template molecule, and carrying out polymerization in the presence of a cross-linking reagent. The template molecules are then removed, leaving the cavities, which will then serve as “molds” to capture the target biomolecule. Molecular imprints have been used for chromatographic separation, immunoassays, chemosensors, and catalysis (Wulff, 1998, supra).

As noted in U.S. Pat. Nos. 6,458,599 and 6,680,210 to Huang, assigned to Aspira Biosystems, Inc., and in U.S. Patent Publication No. 2004/0018642 A1 to Huang, the use of molecular imprints to capture biomolecules is quite limited. According to a recent review, two issues “of great importance” that limit the application of conventional molecular imprints are their limited capacity and the heterogeneity of their imprint cavities (Cormack et al. (1999) Reactive and Functional Polymers 41:115-124). When used in an assay to capture a target molecule, it is believed that the random distribution of imprint cavities throughout a conventional molecular imprint limits the access of template molecules to the imprint cavities. The majority of cavities are localized in the interior of the molecular imprint and are less accessible to the template molecule than cavities that are localized at the surface of the imprint. In particular, large molecules that cannot penetrate the matrix material of a molecular imprint can bind only at surface cavities. As also explained in the aforementioned patent documents to Huang, the binding capacity of molecular imprints is also reduced by the random orientation of their cavities. In forming a molecular imprint by conventional techniques, the template molecules are randomly oriented within the matrix. Thus, the corresponding molecular imprint cavities are also randomly oriented. If a particular orientation of an imprint cavity binds a target molecule more efficiently than other orientations, then only the fraction of cavities that are properly oriented will display efficient binding. The random orientation of the cavities, combined with their random distribution throughout the imprint, exacerbates the poor binding capacity of conventional molecular imprints. A further drawback of conventional molecular imprints is that template molecules trapped deep within the imprint matrix may not be removed, and may leak during use. Leakage of the template molecule hinders application of conventional molecular imprints, particularly applications that involve minute amounts of a target molecule or dilute solutions. This shortcoming of conventional molecular imprints has limited their application in the pharmaceutical industry.

In order to overcome the above-described shortcomings of conventional molecular imprints, a conceptually new and unique method of forming and using an imprint material was developed and is described in U.S. Pat. Nos. 6,458,599 and 6,680,210 to Huang, and in U.S. Patent Publication Nos. 2002/0110901 A1, 2002/0164643 A1, 2003/0165882, 2003/0165987 A1, and 2004/0018642 A1, all to Huang. The aforementioned patents and patent publications involve the use of template molecules that correspond to a segment of the targeted biomolecule rather than to the entire biomolecule. The segment to which the template molecule corresponds may be an internal region of the biomolecule or an external (e.g., terminal or side-chain) segment of the biomolecule. The “partial imprint material” so provided thus has cavities corresponding to only a selected segment of the targeted biomolecule, but is nevertheless capable of capturing the entire biomolecule. The partial imprint material has been established to provide significant advantages over prior molecular imprinting technologies such as those described above. Unlike conventional molecular imprint materials, partial imprint materials do not require a purified sample of a target biomolecule for preparation, since the structure of only one segment of the biomolecule is sufficient to create the cavities in the imprint material. Because they do not require isolation of the biomolecule of interest, partial imprint materials can be prepared in far less time and at a fraction of the cost of conventional molecular imprint materials.

To date, however, there has been no suggestion in the art to use partial imprint materials in a dynamic system, such as an extracorporeal fluid circuit, for removing biomolecules from a flowing body fluid such as whole blood or plasma. In fact, the Huang et al. patents and patent applications note that the ideal conditions for capturing targeted biomolecules using the disclosed partial imprint material are identical to the conditions used for manufacturing the material, i.e.: (1) polymerization conditions, involving a polymerization solvent, an optional polymerization initiator, and a catalyst and/or radiation; or (2) heating to liquefy the matrix material, which is mixed with the template molecules, and which solidifies upon cooling to form the partial imprint material. In an extracorporeal system for treating whole blood or plasma, for example, the use of polymerization conditions and high temperatures would clearly be inappropriate and cause serious problems, destroying vital components of the body fluid and rendering it unsuitable for reinfusion into the patient. Furthermore, it would not be expected that the “partial” cavities of a partial imprint material could effectively capture a substantial amount of a targeted biomolecule in a dynamic system wherein the fluid flow is on the order of 25 mmin to 50 ml/min or greater.

### SUMMARY OF THE INVENTION

In one embodiment of the invention, a method is provided for selectively removing a target biomolecule from a body fluid of a patient, comprising:
passing at least one component of the body fluid through a partial imprint material comprising a matrix composition having a plurality of partial imprint cavities, wherein each partial imprint cavity is of a template molecule that corresponds to a segment of the biomolecule, under conditions in which the partial imprint cavities capture the target biomolecule, thereby depleting the body fluid of the biomolecule.

The biomolecule may be any molecular entity that is associated with or causing an adverse medical condition, disease, or disorder. The body fluid is generally blood, but may also be some other body fluid, e.g., lymph or spinal fluid.

In another embodiment, a method is provided for treating a patient by removing a target biomolecule from the patient's blood, comprising:
(a) continuously removing a flow of blood from the patient;
(b) continuously separating the removed blood into a plasma component and a cellular component;
(c) continuously passing at least one of the plasma component and the cellular component through a partial imprint material comprising a matrix composition having a plurality of partial imprint cavities, wherein each partial imprint cavity is of a template molecule that corresponds to a segment of the target biomolecule, under conditions in which the partial imprint cavities capture the biomolecule, thereby producing a treated plasma component and/or a treated cellular component; and
(d) continuously returning the treated plasma component and/or the treated cellular component a to the patient's body.

In still another embodiment, an apparatus is provided for treating a patient by removing target biomolecules from the patient's blood, comprising:
means for removing a continuous flow of blood from the patient;
means for continuously separating the removed blood into a plasma component and a cellular component;
means for continuously passing at least one of the plasma component and the cellular component through a partial imprint material comprising a matrix composition having a plurality of partial imprint cavities, wherein each partial imprint cavity is of a template molecule that corresponds to a segment of the target biomolecule, under conditions in which the partial imprint cavities capture the biomolecule, to thereby produce a treated plasma component and/or a treated cellular component; and
means for returning the treated plasma component and/or the treated cellular component to the patient.

In a further embodiment, a method is provided for treating a patient suffering from an autoimmune disease, the method comprising:
(a) continuously removing a flow of blood from the patient;
(b) continuously separating the removed blood into a plasma component and a cellular component;
(c) continuously passing at least one of the plasma component and the cellular component through a partial imprint material comprising a matrix composition having a plurality of partial imprint cavities, wherein each partial imprint cavity is of a template molecule that corresponds to a segment of an autoantibody associated with the autoimmune disease, under conditions in which the partial imprint cavities capture the autoantibody, thereby producing a treated plasma component and/or a treated cellular component; and
(d) continuously returning the treated plasma component and/or the treated cellular component to the patient's body.

The autoimmune disease may be, for example, Addison's disease, Crohn's disease, IDDM, Grave's disease, Hashimoto's thyroiditis, myasthenia gravis, autoimmune gastritis, pernicious anemia, multiple sclerosis, Sjögren's syndrome, autoimmune hemolytic anemia, thrombocytopenia purpura, psoriasis, pemphigus vulgarus, bullous pemphigoid, epidermolysis bullosa acquisita, cutaneous lupus erythematosis, systemic lupus erythematosis SLE, scleroderma, rheumatoid arthritis, or an antiphospholipid/cofactor syndrome.

Numerous other adverse medical conditions, disorders, and diseases can be treated using the method and apparatus of the invention as will be discussed in detail infra.

### BRIEF DESCRIPTION OF THE DRAWING

FIG. 1 schematically illustrates a modified dialysis system that can be used in conjunction with the method of the invention when the body fluid treated is whole blood.

### DETAILED DESCRIPTION OF THE INVENTION

Before describing the present invention in detail, it is to be understood that this invention is not limited to specific fluids, biomolecules, cells or device structures, as such may vary. It is also to be understood that the terminology used herein is for the purpose of describing particular embodiments only, and is not intended to be limiting.

It must be noted that, as used in this specification and the appended claims, the singular forms “a,” “an,” and “the” include plural referents unless the context clearly dictates otherwise. Thus, for example, the term “biomolecule” refers to a single biomolecule or to a plurality of biomolecules that can be the same or different, the term “partial imprint cavity” refers to a single such cavity or a plurality of such cavities that can be the same or different, the term “autoantibody” refers to a single autoantibody, a plurality of identical autoantibodies, or two or more different autoantibodies, and the like.

Accordingly, the invention makes use of a “partial imprint material” for removing a target biomolecule from a patient's body fluid. The partial imprint material is composed of a matrix composition having partial imprint cavities that correspond in three-dimensional structure to a segment of a target biomolecule, but not to the entire biomolecule, such that the segment physically “fits” into the corresponding cavity. Each cavity can, accordingly, serve as a mold capable of capturing a segment of the target molecule, and thus bind the entire target molecule as well. The binding will thus involve physical entrapment but may also involve other types of binding, e.g., hydrogen bonds, ionic bonds, covalent bonds, or van der Waals associations. The segment to which a cavity corresponds may be an internal region of a target molecule or an external (e.g., terminal or side-chain) segment of the target molecule. The term “partial imprints” refer to the aforementioned cavities per se.

The terms “biomolecule” and “biological molecule” are used interchangeably herein to refer to any organic molecule that is, was, or can be a part of a living organism, regardless of whether the molecule is naturally occurring, recombinantly produced, or chemically synthesized in whole or in part. The terms encompass, for example, amino acids, peptidic molecules, such as oligopeptides, polypeptides and proteins and monosaccharides, as well as nucleotides, oligomeric and polymeric species, such as oligonucleotides and polynucleotides, saccharides such as disaccharides, oligosaccharides, polysaccharides, mucopolysaccharides or peptidoglycans (peptido-polysaccharides) and the like. The terms also encompass antibodies, antigens, ribosomes, enzyme cofactors, pharmacologically active agents, and the like. It is understood that a biomolecule may be attached to a living cell, and that the immobilization of the biomolecule can immobilize the cell. The term “target biomolecule” refers to a known biomolecule to be removed from a patient's body fluid.

In one embodiment, the invention provides a method for removing a target biomolecule from a patient's body fluid by bringing the body fluid into contact with a partial imprint material as alluded to above. Contact of the body fluid and the cavities of the partial imprint material can be accomplished either by placing the imprint material within the body or by using an extracorporeal fluid circuit in which the body fluid is removed from the body, circulating past the imprint material, and then returned to the body. These treatment processes can, if desired, take place when the concentration of a particular target biomolecule (e.g., an autoantibody) reaches a certain level, and be stopped when the concentration is reduced to a certain level. Of course, the levels will vary with the nature and stage of the disease, as well as the characteristics of the individual patient.

In general, the method involves removing a volume of body fluid from the patient and treating the body fluid using a system external to the patient's body. The fluid flow in such an extracorporeal fluid circuit is typically on the order of 25 ml/min to 50 mmin or higher. The treated body fluid from which the target molecule has been removed, or at least substantially removed, is ultimately re-introduced into the patient's body. The method removes at least 85 wt. %, preferably at least 90 wt. %, more preferably at least 95 wt. %, and most preferably at least 99 wt. % of the target biomolecule. Generally, the body fluid is blood, although other body fluids are contemplated as well, e.g., lymph and spinal fluids. With blood, the method may be used to treat whole blood or a blood component. For example, the blood may be separated into cellular and plasma components, with one or both of the components being treated with the present method, the components combined following treatment, and the treated fluid then re-introduced into the patient's body.

The method of the invention may, accordingly, be carried out using a modified version of a device that enables removal and extracorporeal treatment of a body fluid such as whole blood, plasma, lymph, or cerebral spinal fluid. One such device is a dialysis machine. As is widely understood, dialysis involves the separation of materials in a liquid based on a difference in the rate of diffusion of the materials through a semipermeable membrane into a second liquid, the dialysate. In patients with reduced or no kidney function, dialysis of the blood, termed “hemodialysis,” is used to remove urea and other waste products from the blood, while blood cells and large proteins are retained. The blood is dialyzed against dialysate that contains electrolytes (e.g., Ca²⁺, Mg²⁺, Na⁺, K⁺, Cl⁻) in amounts selected to maintain proper blood electrolyte concentrations. The dialysis equipment is connected to the patient through a surgically created fistula, which draws blood from an artery and returns it to a vein.

Dialysis machines are in routine use at thousands of clinics throughout the world. Hundreds of thousands of patients are treated in the United States alone. Methods to control blood flow, remove air bubbles, and maintain proper electrolyte balance, blood sugar, oxygenation, temperature, sterility, and other vital factors during dialysis are well known and established in the art. Surgical techniques to create an arterial-venous fistula, from which blood is taken into the dialysis machine and then returned to the body, are also very well established.

In carrying out the method of this invention, existing dialysis systems can be utilized with the dialyzer replaced by a treatment chamber containing a partial imprint material selected to remove a particular biomolecule. In this way, when blood flows through the chamber, the partial imprint material removes one or more target biomolecule from the blood. The imprint material may be in the form of individual beads, disks, ellipses, fibers, sheets, or other regular or irregular shapes contained within the chamber. Preferred shapes are designed to maximize the surface area of the imprint material within the chamber, such that the blood flowing through the chamber contacts as much of the imprint material as possible. The imprint material can also be in the form of a coating on the interior of one or more lengths of tubing through which the blood flows. In the latter embodiment, the tubing is preferably coiled or otherwise convoluted or bent, in order to maximize the amount of imprint material contacted by the blood flowing through the chamber.

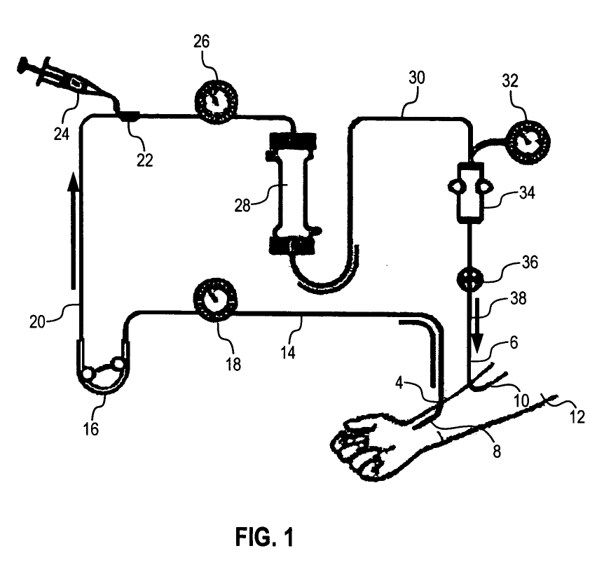

A suitable apparatus for use in the method of the present invention is illustrated schematically in FIG. 1. The apparatus 2 is in the form of a fluid circuit having a blood intake terminus 4 and a blood-returning terminus 6, with each terminus in the form of a cannula, i.e., a blood intake cannula 8 and a blood returning cannula 10. During use, blood is withdrawn from the patient 12 at the blood intake terminus 4 into tubing 14 by means of a blood flow pump 16. The blood is drawn through a first pressure monitor 18 operatively connected to a sensor (not shown). The pump then feeds the withdrawn blood through tubing 20 in which is present a valve 22, which, when open, allows introduction of one or more useful components into the flowing blood, e.g., anticoagulants such as heparin and sodium citrate. The one or more components may be introduced via a syringe 24, as shown, or from a container such as a plastic bag operatively connected to the valve 22. Also present along the tubing 20 is a second pressure monitor 26 with a sensor (not shown) operatively attached thereto, such that the interior pressure can be determined immediately prior to entry of the blood into the treatment chamber 28. The treatment chamber, as explained above, contains a partial imprint material that captures and thus removes one or more types of components from the patient's blood. The blood flows out of the chamber into tubing 30, in which is present a third pressure monitor 32 operatively connected to a sensor (not shown). After passing through the air bubble trap 34 coupled to air bubble sensor 36, the treated blood is returned to the patient through tubing 38 via blood returning cannula 10 at the blood-returning terminus 6 of the apparatus. The air bubble trap 34 and air bubble sensor 36 are used as they are in conventional dialysis systems, i.e., during priming of the apparatus with saline, to ensure that fluid is being circulated and recalculated without any air in the circuit.

Another type of apparatus that can be used in conjunction with the present method is a chemical apheresis system, either a plasmapheresis system or a cytapheresis system, or a device configured so as to carry out both plasmapheresis and cytapheresis. As is understood in the art, plasmapheresis involves the extracorporeal manipulation, depletion and/or removal of certain soluble or suspended components in the plasma portion of the blood, after which the blood so treated is reinfused into the patient to induce a desired clinical effect. Plasmapheresis has been performed in vivo using therapeutic plasma exchange (TPE), immunoadsorption (IA), membrane differential filtration (MIF), and other means. Cytapheresis differs from plasmapheresis in that it involves the extracorporeal manipulation or depletion and/or removal of various circulating or marrow-bound cellular elements in the blood (red cells, white cells, stem cells or platelets) or specific subpopulations of these cells in order to induce a desired clinical effect.

An apheresis-type apparatus, as with the modified dialysis device of FIG. 1, enables continuous, on-line treatment of a patient's blood by removal of a biomolecule therein. The device of FIG. 1, however, is used to remove, treat, and reinfuse whole blood. In this type of apparatus, the whole blood initially withdrawn from the patient via a blood intake terminus is separated into cell concentrate and blood plasma. In plasmapheresis, the separated plasma is be passed through a treatment chamber containing a partial imprint material as described herein, so that one or more target biomolecules within the plasma is removed as a result of having been “captured” by the imprint material. In cytapheresis, it is the cell concentrate that is contacted with a partial imprint material so that one or more target components are removed therefrom. It may be desirable, in some cases, to incorporate both a plasmapheresis circuit and a cytapheresis circuit into a single apparatus. In any of these embodiments, the plasma and cell concentrate, either or both of which having been treated by contact with the partial imprint material, are ultimately combined and reinfused into the patient. Suitable apheresis devices are described, by way of example, in U.S. Pat. No. 5,098,372 to Jonsson and U.S. Pat. No. 5,112,298 to Prince et al., and in “Ig-Therasorb® Immunoadsorption,” published on the web in October 2000 by PlasmaSelect AG (Teterow, Germany).

Care must be taken to select biocompatible materials for the fluid circuit and treatment chamber in any device used to carry out the present method; such materials are well known in the art of dialysis and apheresis. When the cavities of the partial imprint material in the treatment chamber become saturated such that they are no longer capable of capturing the targeted biomolecules, the partial imprint material can be replaced. Alternatively, the saturated imprint material can be “recycled” by means of a treatment that removes captured biomolecules so as to regenerate the partial imprint cavities, enabling reuse of the material in the treatment chamber of the device. Suitable treatments for removing the captured biomolecules include, without limitation, incubation in a chaotropic reagent such as urea or guanidine, and diffusion.

The partial imprint material is composed of a matrix composition that is preferably capable of undergoing a physical change from a fluid state to a semi-solid or solid state, in order to facilitate manufacture of the material by solidifying the matrix composition around selected template molecules and then removing the template molecules from the solidified matrix. Examples of such matrix materials include heat-sensitive hydrogels such as agarose, polymers such as acrylamide, and cross-linked polymers. Other suitable matrix materials and preparation thereof are described infra.

The imprint compositions of the invention may take a variety of different forms. For example, they may be in the form of individual beads, disks, ellipses, or other regular or irregular shapes (collectively referred to as “beads”), or in the form of sheets. Each bead or sheet may comprise imprint cavities of a single template molecule, or they may comprise imprint cavities of two or more different template molecules. In one embodiment, the imprint composition comprises imprint cavities of a plurality of different template molecules arranged in an array or other pattern such that the relative positions of the imprint cavities within the array or pattern correlate with their identities, i.e., the identities of the template molecules used to create them. Each position or address within the array may comprise an imprint cavity of a single template molecule, or imprint cavities of a plurality of different template molecules, depending upon the application. Moreover, the entire array or pattern may comprise unique imprint cavities, or may include redundancies, again depending upon the application.

As discussed above, the template molecule used to make the imprint corresponds to a segment of a target biomolecule of interest. Biomolecules that can be captured using the method of the invention include any type of biomolecule from which a template molecule can be designed and constructed according to the principles set forth by Huang, in, for example, U.S. Patent Publication No. 2004/0018642. Generally, the biomolecules will be: peptidic, including oligopeptides, polypeptides, proteins, and the like; nucleotidic, including oligonucleotides, polynucleotides, DNA, RNA, and the like; saccharidic, including oligosaccharides and polysaccharides; or lipidic, including lipids per se as well as lipoproteins and lipopolysaccharides. Specific classes of target biomolecules thus include, without limitation, cytokines, hormones, growth factors, enzymes, cofactors, ligands, receptors, antibodies, carbohydrates, steroids, therapeutics, antibiotics, and even larger structures such as viruses or cells, and other macromolecular targets that will be apparent to those of skill in the art.

The target biomolecules may be in the form of the naturally occurring molecules found in a “normal” individual's body fluid, or they may be modified in some way, for example, by enzymatic or other chemical reactions. The target biomolecules may also be manufactured by the body in an altered form as a result of a genetic abnormality.

The present invention does not place any size limitations on the target biomolecules to be captured and removed from a patient's body fluid. In fact, the target biomolecule may be present on the surface of a cell, in which case capture of the target biomolecule by a partial imprint cavity also results in capture of the cell. In certain cases, removal of a particular cell type can be therapeutically beneficial, as in the removal of specifically targeted cancer cells from a patient's blood. Other disorders and diseases that can be treated in this way will be apparent to those of ordinary skill in the art and/or described in the pertinent texts and literature.

The identities of the structural moieties that represent the segments of target biomolecules that correspond to the cavities of the partial imprint material used herein will depend upon the nature of the biomolecule, and may include regions of primary, secondary, and/or tertiary structure of the biomolecule. With polypeptides, the structural moieties may be individual amino acids in the polypeptide, or, alternatively, if the polypeptide has several structural domains, as is often the case with enzymes and antibodies, the structural moieties may be the individual structural domains. For example, an antibody may be viewed as being composed of individual amino acids, or Fab and Fc structural domains, or alternatively, Fab′ and Fc structural domains, etc., depending upon the proteolytic enzymes used to digest the antibody. A glycosylated polypeptide may be viewed as being composed of individual amino acids or structural domains as described above and/or saccharide or oligosaccharide structural moieties. A polynucleotide may be viewed as being composed of individual nucleotide structural moieties.

Structural moieties may also be regions of secondary structure, such as regions of α-helix, β-sheet, β-barrel, etc. of proteins or regions of A-form helix, B-form helix, Z-form helix, triple helix, etc. of polynucleotides.

For non-polymeric biomolecules such as, for example, antibiotics, the structural moieties may be the various core groups composing the antibiotic. For example, polygene antibiotics such as amphotericin B and nystatin may be viewed as being composed of polyene macrocycle and saccharide structural moieties.

Use of biomolecules to prepare template molecules is described in detail in U.S. Patent Publication No. 2004/0018642, referenced supra. As noted therein, the template molecule may correspond to any segment of the target biomolecule, including an internal region, a terminal region, or a modifying molecule such as a polysaccharide of a glycoprotein. Preferably, the segment of the target biomolecule is of sufficient size that the biomolecule forms a tight complex with the molecular imprint. Also preferably, the segment of the biomolecule is sufficiently unique that the molecular imprint is selective for the biomolecule. For instance, if the biomolecule is to be captured from a complex mixture, a preferred template molecule corresponds to a segment of the target biomolecule that uniquely defines that biomolecule over other biomolecules in the mixture. Such a unique segment of the biomolecule can be determined by comparing the structure of the biomolecule with the structures of known biomolecules of the complex or by statistical analysis. When the biomolecule is a polypeptide, a template molecule consisting of a sequence of seven amino acids can provide a molecular imprint that is highly selective for the target biomolecule.

In general, template molecules can correspond to any segment of the target biomolecule, as long as the template does not correspond to the entire biomolecule. If the target biomolecule consists of n identifiable structural units, then the segment of the biomolecule to which the template corresponds can consist of from 1 up to (n−1) of those structural units. Preferably, the structural units of the biomolecule to which the template molecule corresponds are contiguous within the primary structure of the biomolecule. If one of skill in the art can identify a terminus or termini in the primary structure of the biomolecule, then a preferred template molecule corresponds to a template that includes a terminus of the biomolecule. Alternatively, the segment of the biomolecule to which the template molecule corresponds can be expressed in size as a fraction of the size of the entire biomolecule. For example, template molecules can correspond to a segment of the biomolecule that consists of from 1% to 5%, from 1 to 10%, from 1 to 15%, from 1 to 20%, from 1 to 25%, from 1 to 30%, from 1 to 35%, from 1 to 40%, from 1 to 50%, from 1 to 60%, from 1 to 70%, from 1 to 80%, from 1 to 90%, from 1 to 95%, or from 1 to 99% of the structure of the entire biomolecule. Preferably, template molecules have a primary structure that corresponds to a contiguous segment of the primary structure of the biomolecule. If the biomolecule is a polymer, the structure of the template molecule can correspond to a sequence of monomers from the polymer.

In instances where the target biomolecule is a polymer, the segment of the polymer to which the template molecule corresponds can also be expressed as a range of monomer units of the polymer. If the polymer consists of n monomer units, then the segment of the polymer, to which the template molecule corresponds, can consist of up to (n−1) monomer units. Alternatively, the segment of the polymer can consist of from 1 to 50 monomer units, from 2 to 40 monomer units, from 3 to 30 monomer units, from 3 to 15 monomer units, from 3 to 10 monomer units, from 4 to 10 monomer units, from 4 to 9 monomer units, from 4 to 8 monomer units, from 4 to 7 monomer units, or from 5 to 7 monomer units. If the polymer is branched, then the segment can be branched or unbranched. Preferably, the segment of the polymer is a contiguous sequence of monomers from the primary structure of the polymer.

If the target biomolecule is a polypeptide, the template molecule can correspond to a segment of the polypeptide that consists of a sequence of amino acids selected from the primary sequence of the polypeptide. For instance, if the polypeptide has a length of n amino acids, the segment of the biomolecule to which the template molecule corresponds can consist of up to (n−1) amino acids of the primary structure of the polypeptide. Alternatively, the segment of the polypeptide can consist of a range of amino acids from the primary structure of the polypeptide consisting of from 1 to 50 amino acids, from 2 to 40 amino acids, from 3 to 30 amino acids, from 3 to 15 amino acids, from 3 to 10 amino acids, from 4 to 10 amino acids, from 4 to 9 amino acids, from 4 to 8 amino acids, from 4 to 7 amino acids, or from 5 to 7 amino acids. Preferred segments of the biomolecule are those that consist of a continuous sequence of amino acids from the primary structure of the polypeptide. When the biomolecule is a polypeptide, the preferred template molecule corresponds to the contiguous sequence of seven amino acids at the carboxy terminus of the polypeptide. Template molecules that correspond to the amino-termini are less preferable because polypeptides from biological sources often have heterogeneous modifications at their amino-termini.

When the biomolecule is a polypeptide modified with a polysaccharide, the template molecule can be a polysaccharide having a sequence of saccharides selected from the primary sequence of the polysaccharide. If a contiguous polysaccharide component of the polypeptide contains n saccharide units, then the selected sequence of saccharides can contain up to n saccharides. Alternatively, the selected sequence can contain from 1 to 50 saccharides, 2 to 40 saccharides, 3 to 30 saccharides, 3 to 15 saccharides, 3 to 10 saccharides, 4 to 10 saccharides, 4 to 9 saccharides, 4 to 8 saccharides, 4 to 7 saccharides, or 5 to 7 saccharides. If a polysaccharide component of the polypeptide is branched, the template molecule can also be branched. A preferred template molecule corresponds to a contiguous sequence of saccharide units from the polysaccharide, whether branched or unbranched. The template molecule can also correspond to a hybrid structure selected from the primary structure of polypeptide consisting of at least one amino acid and at least one saccharide.

When the biomolecule is a polynucleotide, the template molecule can be an oligonucleotide having a sequence of nucleotides selected from the primary sequence of the polynucleotide. If the polynucleotide has n nucleotides, then the selected sequence of nucleotides can have a length from 1 to (n−1) nucleotides. Alternatively, the selected sequence can contain from 1 to 50 nucleotides, 2 to 40 nucleotides, 3 to 30 nucleotides, 3 to 15 nucleotides, 3 to 10 nucleotides, 4 to 10 nucleotides, 4 to 9 nucleotides, 4 to 8 nucleotides, 4 to 7 nucleotides, or 5 to 7 nucleotides. Preferably, the selected sequence is a contiguous sequence of nucleotides from the primary sequence of the polynucleotide.

When the biomolecule is any type of polymer, the selected sequence of monomers for the structure of the template molecule can be chosen from an internal region of the polymer or from a terminus of the polymer. If the polymer has unique termini, the sequence of monomers can also be chosen from any terminus of the polymer. If the biomolecule is polypeptide, the preferred template molecule for the present invention corresponds to the sequence of amino acids at the carboxy terminus of the polypeptide.

For biomolecules that are non-polymeric such as, for example, antibiotics, the template molecule preferably corresponds to a structural feature that uniquely identifies the antibiotic. For example, if several antibiotics differ in structure from one another by the identity of a single substituent (e.g., a sugar residue, a lipid moiety, etc.), then a molecular imprint prepared with a template molecule that corresponds to that unique feature can be used to specifically capture that antibiotic from a complex mixture of related antibiotics. For example, the antibiotic amphotericin B (AmB) can be selectively captured from a mixture of AmB and amino sugar derivatives thereof with a molecular imprint prepared with a template that corresponds to the amino sugar moiety of amphotericin.

If mixtures of several distinct biomolecules are to be captured, the template can correspond to a common structural feature. For example, a mixture of AmB and amino sugar derivatives thereof can be captured with a molecular imprint prepared with a template molecule that corresponds to their common polyene core.

Use of template molecules to manufacture partial imprint materials in the present method is described in detail in U.S. Patent Publication No. 2004/0101465, referenced supra. A general method for preparing a partial imprint material involves admixing a heat-sensitive compound or composition with a template molecule under conditions in which the heat-sensitive compound liquefies. The heat source is then removed, and as the liquid cools, it solidifies to form a matrix containing template molecules embedded therein. Many heat-sensitive compounds that can be used to make imprint compositions according to the invention are known in the art and include, by way of example and not limitation, hydrogels such as agarose, gelatins, moldable plastics, etc. Examples of other suitable hydrogels are described in U.S. Pat. Nos. 6,018,033, 5,277,915, 4,024,073, and 4,452,892. Preferably, the temperature at which the heat-sensitive compound liquefies will be in a range that does not destroy or otherwise substantially degrade the template molecule.

In analogous methods, the matrix composition may be manufactured from compounds or compositions that undergo a chemically induced or radiation-induced liquid-to-solid change of state. In this case, the template molecule and the compound or composition that will form the matrix material are admixed and treated chemically and/or with radiation to solidify the matrix material with the template molecules embedded therein. Examples of these types of compounds styrene, methyl methacrylate, 2-hydroxyethyl methacrylate, 2-hydroxyethyl acrylate, methyl acrylate, acrylamide, vinyl ether, vinyl acetate, divinylbenzene, ethylene glycol dimethacrylate, ethylene glycol diacrylate, pentaerythritol dimethacrylate, pentaerythritol diacrylate, N,N′-methylenebisacrylamide, N,N′-ethylenebisacrylamide, N,N′-(1,2-dihydroxyethylene)bis-acrylamide, trimethylolpropane trimethacrylate, vinyl cyclodextrin, and polymerizable cyclodextrin. Further examples of polymerizable compounds that are useful for the preparation of molecular imprints can be found in U.S. Pat. No. 5,858,296. The preferred polymerizable substance is acrylamide. If necessary, an initiator for the polymerization of the polymerizable compound is included. Optionally, the template molecule can be covalently bound to the polymerizable compound, or the two can be allowed to form non-covalent complexes. Polymerization can by started by addition of a catalyst and/or irradiation.

After formation of the partial imprint material, the template molecule is removed by diffusion, by incubation in a chaotropic reagent such as urea or guanidine, or by other techniques known to those of skill in the art.

Once the matrix is in a solid or semi-solid state, the molecular imprints can be processed to take on a variety of shapes. Usually, the molecular imprint will initially take on the same shape as the container used to create the matrix. However, any shape that might be useful for capturing target biomolecules is possible. For example, they may be in the form of individual beads, disks, ellipses, or other regular or irregular shapes (collectively referred to as “beads”), or in the form of sheets. Beads can be formed by grinding a rigid matrix or by suspension and dispersion techniques, as is well known in the art.

Also within the scope of the present invention are methods of using molecular imprints to capture target biomolecules. Molecular imprints useful for capturing biomolecules can are prepared as described above. To capture a target biomolecule or a mixture comprising the biomolecule is contacted with the molecular imprint under conditions in which the biomolecule binds the imprint. A target biomolecule “binds” a cavity if it becomes entrapped or immobilized within the cavity in a specific manner such that it is specifically captured from the body fluid undergoing treatment. For capture, the imprint compositions may be disposed within a housing to create a chromatography column, or used batch-wise. Also preferably, the conditions for contacting the target biomolecule with the imprint are similar to or identical to the conditions under which the imprint was formed.

The choice of conditions depends on the target biomolecule and the template molecule that corresponds to a portion of the biomolecule. When the biomolecule is a protein and the template molecule corresponds to sequence of amino acids of the protein, the preferred capture conditions are often denaturing. However, when a template molecule corresponds to a large fragment of a protein, such as a pepsin fragment of an immunoglobulin, then native imprinting and capture conditions will often yield superior results. When the biomolecule is a double-stranded polynucleotide, the preferred capture conditions are native conditions that allow the biomolecule to maintain its native structure. When the biomolecule is a single-stranded polynucleotide, the capture conditions may be native or denaturing conditions. One of skill in the art will recognize whether native or denaturing conditions are appropriate. In situations where the choice of imprinting and capture conditions is not clear, the molecular imprint compositions of the present invention can be prepared so efficiently and inexpensively that a series of conditions can be assayed to determine the ideal conditions.

The exact conditions to retain a native or denatured structure are well known and will be apparent to those of skill in the art. For instance, denaturing conditions for polypeptides can include SDS, urea, guanidine, or any other protein denaturant known to those of skill in the art. Denaturing conditions for polynucleotides can include high temperature, formamide, high ionic strength, and other conditions known to those of skill in the art.

The selection of the imprint geometry can be done by a variety of means. With antibodies, there is frequently a unique sequence of amino acids suitable for high specificity selection and located on the exterior or otherwise readily accessible region of the molecule. Finding a suitable short amino acid sequence for an antibody can be done by experimental means. For example, DNA sequence data can be used to determine the amino acid sequence. Short sections of this sequence, in the range of 5 to 12 amino acids, can then be selected. Specificity can be analyzed by scanning the proteome to ensure uniqueness of the sequence. Experimental methods include the synthesis of these small peptides to generate imprint cavities. The specificity of these imprints for the target capture materials can then be tested against the whole fluid samples as well as single amino acid variants. Peptides that give the best specificity and capture properties could then be chosen for used for capture therapy. It will be appreciated that this method is not limited to antibodies, but can readily extend to proteins, modified proteins, or peptidic macromolecules.

Representative uses of the present method are set forth below.

Certain peptidyl compounds, including oligopeptides, polypeptides, and proteins, are known to form deposits that are associated with various disorders and diseases. One of the best known examples of such a disease is sickle cell anemia, which is caused by a single inherited point mutation in the gene coding for adult hemoglobin. The mutated protein (hemoglobin S) in the deoxygenated form is slightly less soluble than normal hemoglobin. As a result, it polymerizes and precipitates as fibers or crystals that distort and ultimately destroy the enclosing erythrocyte, with potentially severe medical consequences to the affected individual (see, e.g., Noguchi C. T. et al. (1985) “Sickle hemoglobin polymerization in solution and in cells,” Ann Rev Biophys Biophys Chem 14:239-63; and Poyart, C. et al. (1996), “Hemolytic anemias due to hemoglobinopathies,” Mol Aspects Med 17:129-42). he methodology of the invention can accordingly be implemented in the treatment of sickle cell anemia by removing the mutated protein from the body fluid of a patient afflicted with the disease.

Peptidyl molecules can also represent key components of plaques and deposits associated with various medical pathologies. Such biomolecules include, without limitation: cystic fibrosis transmembrane conductance regulator (“CFTR”) protein, crystallization of which is associated with cystic fibrosis (see Berger et al. (2000), “Differences Between Cystic Fibrosis Transmembrane Conductance Regulator and H is P in the Interaction with the Adenine Ring of ATP,” J Biol Chem 275:29407-29412); phospholipases, which form Charcot-Leyden crystals associated with asthma, eosinophilic bone granuloma, eosinophilic pneumonia, and granulocytic leukemia (see Reginato and Kumik (1989), “Calcium Oxalate and Other Crystals Associated with Kidney Diseases and Arthritis,” Semin Arthritis Rheum 18:198-224); cystine, which forms crystal deposits in bone marrow (associated with rickets and synovitis), the renal tubule and gastrointestinal tract (associated with cystinuria), and a variety of other body tissues, including the kidneys, eyes, and thyroid glands (associated with cystinosis, including the severe form of the disease, nephropathic cystinosis, or Fanconi's syndrome); and hemoglobin, hematoidin, cryoglobulins, and immunoglobulins (associated with hemarthrosis and other joint disorders, cryoglobulinemia, and multiple myeloma). See Gatter and Owen, Jr., “2. Crystal Identification and Joint Fluid Analysis,” in Gout, Hyperuricemia, and Other Crystal-Associated Arthropathies, Eds. Smyth et al. (New York: Marcel Dekker Inc., 1999), pp. 15-28; and Reginato and Kurnik, supra. The present method may be used to remove such pathology-inducing peptidic molecules from the body fluid of a patient suffering from one of the aforementioned disorders.

Alzheimer's disease: Amyloid peptides, particularly β-amyloid, are known to form ordered fibrillar aggregates that comprise the extracellular and cerebrovascular senile plaques associated with Alzheimer's disease. See Han et al. (1995), “The Core Alzheimer's Peptide NAC Forms Amyloid Fibrils which Seed and are Seeded by β-Amyloid: is NAC a Common Trigger or Target in Neurodegenerative Disease?” Chemistry and Biology 2:163-169; Serpell et al. (2000), “Molecular Structure of a Fibrillar Alzheimer's Aβ,” Biochemistry 39:13269-13275; Jarrett and Lansbury (1992), “Amyloid Fibril Formation Requires a Chemically Discriminating Nucleation Event: Studies of an Amyloidogenic Sequence from the Bacterial Protein OsmB,” Biochemistry 31(49):12345-12352; and Jarrett et al. (1993), “The Carboxy Terminus of the β-Amyloid Protein is Critical for the Seeding of Amyloid Formation: Implications for the Pathogenesis of Alzheimer's Disease,” Biochemistry 32:4693-4697. Amyloid-β (1-40), amyloid-β (1-42), and amyloid-β (1-43), are peptide fragments of particular interest, and the method of the invention thus targets these biomolecules in one preferred embodiment.

Treatment of Parkinson's disease: Parkinson's disease is a progressive neurodegenerative disorder characterized by muscle tremors, as well as by impaired movement, muscle control, and balance. These symptoms result from destruction of dopamine-producing cells in a region of the upper brain stem known as the substantia nigra. Dopamine from the substantia nigra stimulates regions of the brain important for motor control, particularly the corpus striatia, as well as regions in the frontal lobe responsible for attention and executive control. The progressive loss of dopamine from the substantia nigra leads to a gradual loss of muscle control, and ultimately to muscle rigidity and death. The cause of cell destruction in the substantia nigra is not known for most Parkinson's patients. Genetic factors, injury, and exposure to certain drugs, pesticides, and other chemicals have been implicated in the etiology of Parkinson's disease. The substantia nigra (and sometimes other tissues in the brain and elsewhere) of Parkinson's patients contains fibrous masses called Lewy bodies, which are associated with cell loss. Lewy bodies contain large amounts of the protein α-synuclein. A rare genetic form of Parkinson's disease is caused by mutations in the α-synuclein gene, causing the protein to clump and accumulate. Normally, α-synuclein combines with ubiquitin, which signals it for destruction by proteosomes. This ubiquitinization is mediated by the protein parkin (Chung et al., Nature Medicine 7:1144-1150, 2001). Another rare genetic form of Parkinson's disease is caused by a defect in the parkin gene, causing α-synuclein to build up instead of being broken down. It is likely that the more common forms of Parkinson's disease are also caused by defective α-synuclein, parkin, or associated proteins (Tan et al., Neurology 62:128-131, 2004; Siderowf et al., Ann Intern Med 138:651-658, 2003). These defective proteins are targets for removal from body fluids using the methodology of this invention. In addition, β-amyloid can stimulate α-synuclein production, such that β-amyloid is also a useful target for removal from a patient's body fluid in the treatment of Parkinson's disease.

Prion diseases: The prion diseases, e.g., the class of diseases known as the transmissible spongiform encephalopathies, are also characterized by abnormal protein deposition in brain tissue. In the prion diseases, the abnormal protein deposits are comprised of fibrillar amyloid plaques that are formed primarily from the prion protein (PrP). Such diseases include scrapie, transmissible mink encephalopathy, chronic wasting disease of mule deer and elk, feline spongiform encephalopathy, and bovine spongiform encephalopathy (“mad cow disease”) in animals, and Kuru, Creutzfeldt-Jakob disease, Gerstmann-Struessler-Scheinker disease, and fatal familial insomnia in humans. It has been proposed that a 15-mer amino acid sequence, PrP96-111, is responsible for initiating prion formation in vivo by providing a seed for amyloid fiber formation. See Come et al. (1993), “A Kinetic Model for Amyloid Formation in the Prion Diseases: Importance of Seeding,” Proc Natl Acad Sc. USA 90:5959-5963. The method of the invention can thus be used to treat individuals afflicted with a prion disease by using partial imprints to capture PrP96-111 or other key oligopeptides, peptides, and/or proteins that have a role in initiating prion formation.

Sepsis: Sepsis, also known as toxic shock, is a syndrome characterized by a grossly excessive systemic inflammatory response to a pathogen or toxin. This response can lead to fever or hypothermia, hypotension, excessive coagulation, organ dysfunction and failure, and death. Even when the triggering agent, generally an infection, is eliminated, the destructive hyperinflammatory cascade commonly cannot be controlled. Most current treatments for sepsis do not target the root cause of the disease but rather focus on secondary effects such as blood clotting. In part, sepsis has been difficult to treat because of the massive nature of the infection, rendering traditional antibiotic agents ineffective. For example, the dose of antibiotics necessary to clear a sepsis infection would likely to prove fatal to the patient. Endotoxins, which are lipopolysaccharide complexes found in the outer membranes of Gram-negative bacteria, are potent and common instigators of sepsis. These compounds directly trigger inflammation, fever, and coagulation in mammals. While the bacteria themselves usually can be eliminated from the body with antibiotics, endotoxins released by the bacteria remain, and the ensuing sepsis is commonly fatal. This invention can be used to remove these endotoxins from the blood and other body fluids. Partial imprints for the specific polysaccharides present in the lipopolysaccharides can be used to capture and remove the endotoxins, while leaving vital normal sugars and lipids in place. Coagulation, and the reverse process of fibrolysis, is modulated by dozens of proteins and small molecules. During sepsis, fibrolysis is inhibited and coagulation promoted, leading to excessive clotting, which can decrease circulation to vital organs. One factor controlling clotting and fibrolysis is, for example, activated protein C, which may be deficient during sepsis. This protein promotes fibrolysis and inhibits coagulation by the inactivation of factors Va and VIIIa, which are essential for converting prothrombin to thrombin. The present invention can be used to capture and remove factors Va and VIIIa from the blood of a sepsis patient, to restore the normal balance between coagulation and fibrolysis. Factors Va and VIIIa are proteins, as are most of the pro-coagulation factors. Other proteins that promote coagulation can also be captured and removed from the blood using this invention.

Age-Related Macular Degeneration (AMD): AMD is a chronic, progressive, degenerative eye disease of unknown etiology. AMD is characterized by progressive loss of central vision, and is the most common cause of blindness in patients over age 65 in the industrialized world. AMD is divided into “Dry” and “Wet” forms depending upon the morphological characteristics associated with the observed eye pathology. Recently, Allikmets and others presented landmark research that implicates a defective gene in the pathogenesis of AMD (Allikmets et al., (1997) Science 277, 1805-1807). The gene, which is located at the 1p21 locus, codes for the manufacture of the ATP Binding Cassette transporter retina-specific (ABCR) protein. This superfamily of proteins is believed to be responsible for energy-dependent transport of various substances across retinal membranes, thus promoting the clearance of the retina's waste by-products of vision. In both rod and cone outer segments, hundreds of pigment disks are shed into the retinal pigment epithelial layer (RPE) of the retina daily. There the RPE cells phagocytize the pigment disk material, preparing it for transport across Bruch's membrane to be removed by the adjacent choriocapillary blood supply. The methodology of the invention can be implemented to remove defective ABCR or other chemical entities that have a role in initiating AMD.

Autoimmune diseases: Rheumatoid arthritis is an autoimmune disease of unknown etiology characterized by painful inflammation of one or more joints. The inflammation is usually accompanied by progressive destruction of cartilage and bone. Most individuals with rheumatoid arthritis have the protein termed “rheumatoid factor” in their blood and synovial fluids. Rheumatoid factor (RF) is a pathological antibody that binds to normal antibodies, specifically immunoglobin G (IgG), to form an immune complex. The immune complex formed by RF is thought to cause or exacerbate inflammation. RF is also common in people with the autoimmune diseases systemic lupus erythematosis and Sjogren's disease. Capture and removal of RF or RF-IgG complexes from the blood or synovial fluid of rheumatoid arthritis patients, or other patients with autoimmune diseases that have elevated RF levels, by the methods of this invention can produce an anti-inflammatory effect. If desired, the partial imprint material used in the method can be prepared so as to remove not only RF and RF-IgG complexes but also antigens that contribute to the symptoms of rheumatoid arthritis.

Grave's disease, characterized by bulging eyes, nervousness, and weight loss, is caused by thyroid hyperactivity. The hyperactivity is due to an autoimmune reaction in which antibodies are produced that mimic thyroid stimulating hormone (TSH). These antibodies attach to TSH receptors on the thyroid gland, stimulating the gland. Capture and removal of the abnormal TSH-mimicking antibodies from the blood by the methods of this invention can safely and effectively treat Grave's disease.

In Hashimoto's thyroidosis, the thyroid gland is attacked by immune cells and auto-antibodies, particularly thyroid peroxidase antibodies (TPOAb). The methods of this invention can be used to capture and remove TPOAb and related auto-antibodies from the blood of patients with Hashimoto's thyroidosis to effectively treat the disease.

As with other autoimmune diseases, systemic lupus erythematosis is associated with the production of antibodies that attack tissues of the body. Common autoantibodies associated with this disease include anti-nuclear antibody (ANA), anti-phospholipid antibody, anti-cardiolipin antibody, anti-histone antibody, and others. Removing ANA or other autoantibodies associated with SLE from the blood of SLE patients can substantially ameliorate the effects of the disease; such removal can be performed using this invention, obviating the need for standard pharmacotherapy with corticosteroids and/or immunosuppressive drugs.

Cancer: The method of the invention can also be used with a partial imprint material designed to capture certain moieties that are associated with and/or exacerbate a malignancy within a patient's body. For example, the cavities of the partial imprint material can be structured to bind tumor necrosis factor alpha (TNF-α) soluble receptors and remove them from a patient's body fluid. Removal of TNF-α soluble receptors from the blood of a cancer patient has been stated to increase the patient's natural killer cell activity and better recognize and attack malignant tissue.

Lipid-Related Diseases and Disorders:

Lipids, particularly sterols and sterol esters, represent an additional class of biomolecules that form pathogenic deposits in vivo. Atherosclerotic plaque (atheroma) and cholesterol emboli are largely composed of cholesterol monohydrate and crystalline cholesteryl esters, including cholesteryl palmitate, oleate, linoleate, palmitoleate, linolenate, and myristate. See North et al. (1978), “The Dissolution of Cholesterol Monohydrate Crystals in Atherosclerotic Plaque Lipids,” Atherosclerosis 30:211-217; Burks and Engelman (1981), “Cholesteryl Myristate Conformation in Liquid Crystalline Mesophases Determined by Neutron Scattering,” Proc Natl Acad Sci USA 78:6863-6867; and Peng et al. (December 2000), “Quantification of Cholesteryl Esters in Human and Rabbit Atherosclerotic Plaques by Magic-Angle Spinning ¹³C-NMR,” Arterioscler Thromb Vasc Biol, pp. 2682-2688. Formation of gallstones is also associated with cholesterol deposits, as gallstones commonly result from the crystallization of cholesterol monohydrate in bile. See Dowling (2000), “Review: Pathogenesis of Gallstones,” Aliment Pharmacol Ther 14 (Suppl. 2):39-46. Cholesterol deposits are associated with a host of additional medical pathologies, including rheumatoid arthritis, systemic lupus erythymatosis, anklosing spondylitis, bone cysts, bone granulomatosis (Erdheim-Chester disease), xanthomas, scleroderma, and paraproteinemia. Reginato and Falasca, “24. Calcium Oxalate and Other Miscellaneous Crystal Arthropathies,” in Gout, Hyperuricemia, and Other Crystal-Associated Arthropathies, supra. In the aforementioned reference, it was also proposed that crystalline deposits of other types of lipids, e.g., fatty acids, are pathogenic as well. See Reginato and Kurnik, supra. The method of the invention can be advantageously implemented in the treatment of diseases and disorders associated with an excess of certain types of lipids, e.g., vascular diseases associated with elevated plasma concentrations of certain lipids, proteins, and other macromolecules that promote accumulations of proteaceous lipid-laden plaques within certain arterial walls. The method can also be used to prevent vascular disease (and ultimately a heart attack) in a patient having elevated levels of such macromolecules by removing the macromolecules prior to deposition of the potentially pathological plaque.

For many applications involving the treatment of vascular diseases, the ability to remove Theologically active macromolecules (RAMs) is of utmost importance. Recent research has demonstrated that fibrinogen, LDL, C-reactive protein, lipoprotein A and other circulating macromolecules have been associated as independent risk factors for the development of vascular disease. These molecules have been documented to precipitate and/or exacerbate the majority of endothelial injury response, vascular smooth muscle cell proliferation and modify extracellular matrix processes and integrated mechanisms associated with acute vascular events as well as participate in “oxidative stress” and “carbonyl stress” that up-regulate vascular injury on the molecular level. The method and system of the present invention are able to suppress this overreaction by depleting the chemical entities associated with the aforementioned risk factors.

Organ transplants: The method of the invention can also be implemented in preventing or ameliorating hyperacute or acute rejection of human donor organ transplanted to a human recipient. Hyperacute rejection would normally occur after a human subject receives a transplanted human organ against which the subject has preformed anti-HLA antibodies. Acute rejection occurs when the recipient of a human organ forms antibodies against that organ after transplant. The present methodology can be used to treat the plasma of the recipient by circulating the plasma through a partial imprint material that can bind and remove a significant portion of the immunoglobulin from the subject.

It is to be understood that while the invention has been described in conjunction with the preferred specific embodiments thereof, the foregoing description is intended to illustrate and not limit the scope of the invention. Other aspects, advantages, and modifications will be apparent to those skilled in the art to which the invention pertains.

All patents, patent applications, journal articles, and other references cited herein are incorporated by reference in their entireties.

1. A method for selectively removing a target biomolecule from a body fluid of a patient, comprising:
passing at least one component of the body fluid through a partial imprint material comprising a matrix composition having a plurality of partial imprint cavities, wherein each partial imprint cavity is of a template molecule that corresponds to a segment of the biomolecule, under conditions in which the partial imprint cavities capture the target biomolecule, thereby depleting the body fluid of the biomolecule.

2. The method of claim 1, wherein the body fluid is selected from blood, lymph fluid, and spinal fluid.

3. A method for treating a patient by removing a target biomolecule from the patient's blood, comprising:
(a) continuously removing a flow of blood from the patient;
(b) continuously separating the removed blood into a plasma component and a cellular component;
(c) continuously passing at least one of the plasma component and the cellular component through a partial imprint material comprising a matrix composition having a plurality of partial imprint cavities, wherein each partial imprint cavity is of a template molecule that corresponds to a segment of the target biomolecule, under conditions in which the partial imprint cavities capture the biomolecule, thereby producing a treated plasma component and/or a treated cellular component; and
(d) continuously returning the treated plasma component and/or the treated cellular component a to the patient's body.

4. The method of claim 3, wherein the biomolecule is a peptidic molecule.

5. The method of claim 4, wherein the biomolecule is an oligopeptide, polypeptide, or protein.

6. The method of claim 5, wherein the biomolecule is a polypeptide.

7. The method of claim 6, wherein the template molecule is an oligopeptide corresponding to a contiguous sequence of the polypeptide.

8. The method of claim 7, wherein the oligopeptide is in the range of about 3 to about 30 amino acids in length.

9. The method of claim 8, wherein the oligopeptide is in the range of about 4 to about 14 amino acids in length.

10. The method of claim 9, wherein the oligopeptide is in the range of about 4 to about 7 amino acids in length.

11. The method of claim 7, wherein the contiguous sequence of the polypeptide is at the C-terminus.

12. The method of claim 3, wherein the target biomolecule is α-synuclein.

13. The method of claim 3, wherein the target biomolecule is an amyloid peptide.

14. The method of claim 13, wherein the biomolecule is selected from the group consisting of amyloid and amyloid-β.

15. The method of claim 14, wherein the biomolecule is amyloid-β.

16. The method of claim 3, wherein the biomolecule is prion protein (PrP).

17. The method of claim 16, wherein the biomolecule is a PrP fragment.

18. The method of claim 16, wherein the biomolecule is PrP96-111.

19. The method of claim 3, wherein the biomolecule is an autoantibody.

20. The method of claim 2, wherein the biomolecule is nucleotidic.

21. The method of claim 20, wherein the template molecule is an oligonucleotide.

22. The method of claim 3, wherein the biomolecule is selected from lipids, lipoproteins, and lipopolysaccharides.

23. The method of claim 3, wherein the biomolecule is a polysaccharide.

24. The method of claim 3, wherein the biomolecule is immunoreactive.

25. The method of claim 3, wherein the biomolecule is present on the surface of a cell, and capture of the biomolecule by a partial imprint cavity results in capture of the cell.

26. The method of claim 3, wherein the matrix composition comprises at least two different partial imprint cavities, wherein each of the at least two different partial imprint cavities corresponds to a different target biomolecule.

27. The method of claim 3, wherein the plasma component is passed through the partial imprint material.

28. The method of claim 3, wherein the cellular component is passed through the partial imprint material.

29. An apparatus for treating a patient by removing target biomolecules from the patient's blood, comprising:
means for removing a continuous flow of blood from the patient;
means for continuously separating the removed blood into a plasma component and a cellular component;
means for continuously passing at least one of the plasma component and the cellular component through a partial imprint material comprising a matrix composition having a plurality of partial imprint cavities, wherein each partial imprint cavity is of a template molecule that corresponds to a segment of the target biomolecule, under conditions in which the partial imprint cavities capture the biomolecule, to thereby produce a treated plasma component and/or a treated cellular component; and
means for returning the treated plasma component and/or the treated cellular component to the patient.

30. The apparatus of claim 29, wherein the biomolecule is a peptidic molecule.

31. The apparatus of claim 30, wherein the biomolecule is an oligopeptide, polypeptide, or protein.

32. The apparatus of claim 31, wherein the biomolecule is a polypeptide.

33. The apparatus of claim 32, wherein the template molecule is an oligopeptide corresponding to a contiguous sequence of the polypeptide.

34. The apparatus of claim 33, wherein the oligopeptide is in the range of about 3 to about 30 amino acids in length.

35. The apparatus of claim 34, wherein the oligopeptide is in the range of about 4 to about 14 amino acids in length.

36. The apparatus of claim 35, wherein the oligopeptide is in the range of about 4 to about 7 amino acids in length.

37. The apparatus of claim 33, wherein the contiguous sequence of the polypeptide is at the C-terminus.

38. The apparatus of claim 30, wherein the target biomolecule is α-synuclein.

39. The apparatus of claim 30, wherein the target biomolecule is an amyloid peptide.

40. The apparatus of claim 39, wherein the biomolecule is selected from the group consisting of amyloid and amyloid-β.

41. The apparatus of claim 40, wherein the biomolecule is amyloid-β.

42. The apparatus of claim 30, wherein the biomolecule is prion protein (PrP).

43. The apparatus of claim 42, wherein the biomolecule is a PrP fragment.

44. The apparatus of claim 30, wherein the biomolecule is an autoantibody.

45. The apparatus of claim 29, wherein the biomolecule is nucleotidic.

46. The apparatus of claim 29, wherein the template molecule is an oligonucleotide.

47. The apparatus of claim 29, wherein the biomolecule is selected from lipids, lipoproteins, and lipopolysaccharides.

48. The apparatus of claim 29, wherein the biomolecule is a polysaccharide.

49. The apparatus of claim 29, wherein the biomolecule is immunoreactive.

50. The apparatus of claim 29, wherein the matrix composition comprises at least two different partial imprint cavities, wherein each of the at least two different partial imprint cavities corresponds to a different target biomolecule.

51. A method for treating a patient suffering from an autoimmune disease,
(a) continuously removing a flow of blood from the patient;
(b) continuously separating the removed blood into a plasma component and a cellular component;
(c) continuously passing at least one of the plasma component and the cellular component through a partial imprint material comprising a matrix composition having a plurality of partial imprint cavities, wherein each partial imprint cavity is of a template molecule that corresponds to a segment of an autoantibody associated with the autoimmune disease, under conditions in which the partial imprint cavities capture the autoantibody, thereby producing a treated plasma component and/or a treated cellular component; and
(d) continuously returning the treated plasma component and/or the treated cellular component to the patient's body.

52. The method of claim 51, wherein the autoimmune disease is selected from Addison's disease, Crohn's disease, insulin-dependent diabetes mellitus (IDDM), Grave's disease, Hashimoto's thyroiditis, myasthenia gravis, autoimmune gastritis, pernicious anemia, multiple sclerosis, Sjögren's syndrome, autoimmune hemolytic anemia, thrombocytopenia purpura, psoriasis, pemphigus vulgarus, bullous pemphigoid, epidermolysis bullosa acquisita, cutaneous lupus erythematosis, systemic lupus erythematosis (SLE), scleroderma, rheumatoid arthritis (RA), and antiphospholipid/cofactor syndromes.

53. A method for treating a patient suffering from a disease or disorder associated with the presence or an excess of a target biomolecule, comprising:
a) continuously removing a flow of blood from the patient;
(b) continuously separating the removed blood into a plasma component and a cellular component;
(c) continuously passing at least one of the plasma component and the cellular component through a partial imprint material comprising a matrix composition having a plurality of partial imprint cavities, wherein each partial imprint cavity is of a template molecule that corresponds to a segment of the target biomolecule, under conditions in which the partial imprint cavities capture the target biomolecule, thereby producing a treated plasma component and/or a treated cellular component; and
(d) continuously returning the treated plasma component and/or the treated cellular component to the patient's body.
